(19) United States
(12) Patent Application Publication
(10) Pub. No.: US 2006/0041308 A1
(43) Pub. Date: Feb. 23, 2006

(54) Posterior chamber implantable intraocular lens

(76) Inventor: Louis D. Nichamin, Brookville, PA (US)

Correspondence Address: HUGH D JAEGER, 1000 SUPERIOR BLVD, SUITE 302, WAYZATA, MN, 553911873, US

(21) Appl. No.: 10/925,115

(22) Filed: Aug. 23, 2004

(51) Int. Cl. A61F 2/16

(52) U.S. Cl. 623/6.43; 623/6.47

(57) ABSTRACT

A posterior chamber implantable intraocular lens having haptics and additional attachment structure of a discontinuous or semi-discontinuous form associated with the haptics. The posterior chamber implantable intraocular lens can be implanted using conventional techniques if the posterior chamber is determined to be suitable and adequate for implantation using conventional techniques, or can be implanted utilizing the additional attachment structure if the posterior chamber is determined to be unsuitable and inadequate for implantation using conventional techniques.

### CROSS REFERENCES TO RELATED APPLICATIONS

None.

### BACKGROUND OF THE INVENTION

1. Field of the Invention

The present invention is for an intraocular lens, and more particularly for a posterior chamber implantable intraocular lens having haptics and additional attachment structure associated with the haptics whereby it can be implanted using conventional techniques in the posterior chamber of the eye if the posterior chamber is determined to be suitable for such implantation, or can be implanted using the additional attachment structure if the posterior chamber is found to be unsuitable.

2. Description of the Prior Art

Prior art posterior chamber implantable intraocular lenses have been available in the art for use using traditional implant techniques. Successful and proper implantation depends on having a posterior chamber which is suitable for fixation of a posterior chamber implantable intraocular lens; i.e., the posterior chamber must not be broken or must not be inadequate for acceptance and support of a posterior chamber implantable intraocular lens. If during an implant procedure it is determined that the posterior chamber is inadequate for implantation of a posterior chamber implantable intraocular lens using traditional implant techniques, the use of a less desirable design or a more complicated suture fixation technique may be considered. What is needed is a posterior chamber implantable intraocular lens which can be suitably implanted into the posterior chamber in general, with possible implantation in the ciliary sulcus or within the remnants of the chamber bag, which offers implantation incorporating traditional implant techniques and which offers an alternate means of implanting should the traditional implant techniques be found to be unfeasible prior to or during an implant procedure. If during or prior to an implant procedure the posterior chamber is found to be inadequate for use incorporating traditional techniques, it would be advantageous if a posterior chamber implantable intraocular lens could still be implanted using alternate means. The present invention provides posterior chamber implantable intraocular lenses which can be implanted using traditional techniques and which can alternately be implanted using enclavement or other techniques.

### SUMMARY OF THE INVENTION

The general purpose of the present invention is to provide a posterior chamber implantable intraocular lens which is implantable by traditional implant techniques, or, alternatively, which is implantable utilizing novel attachment structure should traditional implantation not be possible.

According to one or more embodiments of the present invention, there is provided a posterior chamber implantable intraocular lens including an optical lens and haptics for installation by conventional implantation into the posterior chamber of an eye. Also included is novel attachment structure incorporated with the posterior chamber implantable intraocular lens for attaching the posterior chamber implantable intraocular lens to the posterior surface of the iris, or generally within the posterior chamber, when a broken posterior chamber or inadequate posterior chamber cannot suitably accommodate and fixate the normally used attachment structure of the posterior chamber implantable intraocular lens. The novel attachment structure can be included in several different forms or configurations and can be semi-discontinuous or discontinuous, can be on one or both haptics, can be in the form of an engagement fixture separate from the haptics, can be formed in part by a haptic, or even can be on an aspect of the optical lens itself, and may include a bolt-like or rivet-like fastener.

In one embodiment, the present invention is a posterior chamber implantable intraocular lens including an optical lens with a periphery and diametrically opposed haptics extending from the periphery of the optical lens. Each of the haptics has an outward end, an inward end, an inside aspect, and an outside aspect. Semi-discontinuous attachment structure is situated on each haptic between the outward end and the periphery of the optical lens. The semi-discontinuous attachment structure may include intersecting slots, recesses, or other features which can be on the haptic outside aspect, the haptic inside aspect, or in between the haptic outside aspect and the haptic inside aspect, or at the junction of the haptic with the periphery of the optical lens. If it is determined during an implantation operation that the posterior chamber is broken or inadequate or otherwise unavailable, then posterior iris tissue or other adjoining tissue may be engaged by being urged into or through the slots, recesses, holes, or other features of the semi-discontinuous attachment structure, in an enclavement procedure. The posterior chamber implantable intraocular lens may be formed of a suitable polymer and may be an integral one-piece construction or, in the alternative, may be a multi-piece construction wherein the optical lens has mounting holes in its periphery and separate haptics having inward ends are secured in the mounting holes.

Another embodiment of the present invention is a posterior chamber implantable intraocular lens including an optical lens with a periphery and diametrically opposed haptics extending from the periphery. Each of the haptics has an outward end, an integrally constructed inward end, an inside aspect, and an outside aspect. Semi-discontinuous structures, including configured recesses, are situated on each haptic between the outward end and the periphery of the optical lens, some of which can be on the haptic outside or inside aspect, or in between the outside aspect and the inside aspect, or at the junction of the haptic with the periphery of the optical lens. One configured recess includes a truncated conical portion aligned with and intersecting a cylindrical portion. Another configured recess is similarly constructed, but includes an open slot intersecting both the truncated conical portion and the aligned intersecting cylindrical portion. If it is determined during an implantation operation that the posterior chamber is broken or inadequate or otherwise unavailable, then posterior iris tissue or other adjoining tissue may be engaged by being urged through the slot, if provided, and into the truncated conical portions and aligned intersecting cylindrical portions of the semi-discontinuous attachment structure, in an enclavement procedure. The optical lens could be of multiple member construction having mounting holes in its periphery with the haptics having inward ends secured in the mounting holes. The posterior chamber implantable intraocular lens may be formed of a suitable polymer.

In another embodiment, a posterior chamber implantable intraocular lens includes an optical lens with a periphery, diametrically opposed haptics extending from the periphery, and discontinuous attachment structure associated with the haptics in the form of engagement fixtures extending from the periphery of the optical lens. Attachment structure including several engagement fixtures allows engagement through enclavement of posterior iris tissue in alternative fashion. In one alternative, an engagement fixture comprises first and second extensions having ends spaced from each other and defining a gap. In another alternative, an engagement fixture comprises first and second extensions having opposed ends which overlap either horizontally or vertically in an imbricated contacting or non-contacting arrangement. In yet another alternative, the opposed ends of the extensions can be joined and then a slot and end provided to define a semi-discontinuous attachment structure. The engagement fixtures may be aligned with the diametrically opposed haptics or may be positioned transversely to the diametrically opposed haptics.

In still another embodiment, a posterior chamber implantable intraocular lens includes an optical lens with a periphery, diametrically opposed haptics extending from the periphery, each of the haptics having an extension terminating in an outward end, and projections extending from the periphery, each of the projections having an outward end located near the outward end of a haptic and defining a gap therewith. The diametrically opposed haptics with extensions and the projections together define a pair of opposed and elongated engagement fixtures which constitute yet another discontinuous attachment structure.

In another embodiment, each haptic includes semi-discontinuous attachment structure constituted by a hole through the haptic, and a fastener is provided for extending through the hole to attach to posterior iris tissue when a suitable posterior chamber is unavailable and enclavement is unavailable. The fastener may be selected from amongst barbed clips, nut/bolt assemblies, and capturing head fasteners. The posterior chamber implantable intraocular lens may be formed of a suitable polymer and may be an integral one-piece construction or a multi-piece construction.

In yet another embodiment, each haptic includes semi-discontinuous attachment structure constituted by a recess having an internal flange extending into a cavity to receive iris material for the purpose of enclavement. The diametrically opposed haptics with the recesses, the teachings of which can be utilized in one-piece or multiple-piece constructions define a pair of opposed and elongated engagement fixtures which constitute another semi-discontinuous attachment structure.

The present invention also involves a method of implanting a posterior chamber implantable intraocular lens in a posterior chamber posterior to iris tissue. The method begins by providing a posterior chamber implantable intraocular lens with at least one of the previously described attachment structures. A determination of the suitability and adequacy of the posterior chamber for conventional implantation is made. The provided posterior chamber implantable intraocular lens is then installed in the posterior chamber by conventional implantation if the posterior chamber is determined to be suitable and adequate for conventional implantation; but, if the posterior chamber is determined to be unsuitable and inadequate for conventional implantation, then the posterior chamber implantable intraocular lens is installed in the posterior chamber by attaching it utilizing the provided attachment structure. For example, the posterior chamber may be found to be broken. If the determination is favorable, the posterior chamber implantable intraocular lens is implanted conventionally. If unfavorable, the installing step may be an enclavement procedure or a fastening procedure.

The term “enclavement” as used herein refers to the process of installing the posterior chamber implantable intraocular lens in the posterior chamber of the eye wherein iris tissue is pulled through the intersecting slots, configured recesses, gaps, holes or other provided semi-discontinuous or discontinuous attachment structure associated with the haptics such that the iris tissue is essentially caught by the attachment structure, thus securing the posterior chamber implantable intraocular lens within the posterior chamber.

One significant aspect and feature of the present invention is a posterior chamber implantable intraocular lens which can be implanted into the posterior chamber of the eye even if the posterior chamber is damaged.

Another significant aspect and feature of the present invention is a posterior chamber implantable intraocular lens which can attach to the posterior surface of the iris or be generally secured into the posterior chamber.

Another significant aspect and feature of the present invention is a posterior chamber implantable intraocular lens which can utilize haptics and associated attachment structure for locating the optical lens.

Another significant aspect and feature of the present invention is a posterior chamber implantable intraocular lens which can utilize enclavement for engagement.

Having thus briefly described one or more embodiments of the present invention and having mentioned some significant aspects and features of the present invention, it is the principal object of the present invention to provide a posterior chamber implantable intraocular lens.

### BRIEF DESCRIPTION OF THE DRAWINGS

Other objects of the present invention and many of the attendant advantages of the present invention will be readily appreciated as the same becomes better understood by reference to the following detailed description when considered in connection with the accompanying drawings, in which like reference numerals designate like parts throughout the figures thereof and wherein:

FIG. 1, an embodiment of the present invention, is a top view of a posterior chamber implantable intraocular lens having haptics and semi-discontinuous attachment structure associated with the haptics;

FIG. 2 is a side view of the posterior chamber implantable intraocular lens shown in FIG. 1;

FIG. 3, a first alternative embodiment, is a top view of a posterior chamber implantable intraocular lens having haptics and semi-discontinuous attachment structure associated with the haptics;

FIG. 4 is a side view of the posterior chamber implantable intraocular lens shown in FIG. 3;

FIG. 5 is a top view of a configured recess in a portion of a haptic shown in FIG. 4;

FIG. 6 is a cross section view of a haptic along line 6-6 of FIG. 5;

FIG. 7 illustrates the imbrication of iris material in the configured recess of the haptic of FIG. 6;

FIG. 8, a second alternative embodiment, is a top view of a posterior chamber implantable intraocular lens having haptics and discontinuous attachment structure associated with the haptics;

FIG. 9 is a side view of the posterior chamber implantable intraocular lens shown in FIG. 8;

FIG. 10, a third alternative embodiment, is a top view of a posterior chamber implantable intraocular lens having haptics and discontinuous attachment structure associated with the haptics;

FIG. 11 is a side view of the posterior chamber implantable intraocular lens shown in FIG. 10;

FIG. 12, a fourth alternative embodiment, is a top view of a posterior chamber implantable intraocular lens having haptics and semi-discontinuous attachment structure including fastener devices associated with the haptics;

FIG. 13 is a side view of the posterior chamber implantable intraocular lens shown in FIG. 12;

FIG. 14, a fifth alternative embodiment, is a top view of a posterior chamber implantable intraocular lens having haptics and semi-discontinuous attachment structure including configured recesses associated with the haptics;

FIG. 15 is a side view of the posterior chamber implantable intraocular lens shown in FIG. 14; and,

FIG. 16 is a cross section view of a configured recess along line 16-16 of FIG. 14.

### DETAILED DESCRIPTION OF THE PREFERRED EMBODIMENTS

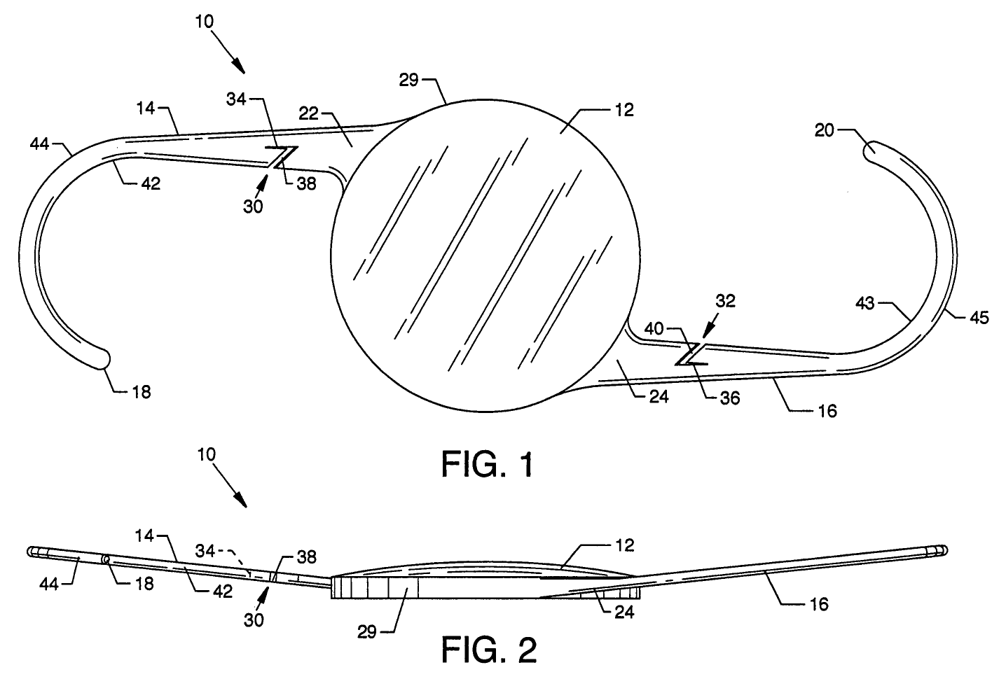

FIG. 1 is a top view, and FIG. 2 is a side view of a posterior chamber implantable intraocular lens 10, including an optical lens 12 and diametrically opposed haptics 14 and 16 with outward ends 18 and 20 and integrally constructed inward ends 22 and 24 wherein the inward ends 22 and 24 of the haptics 14 and 16 intersect and are continuous with the periphery 29 of the optical lens 12. Alternatively, haptics having many similar qualities and features of the haptics 14 and 16 and other haptics herein could be haptics of a multiple-piece posterior chamber implantable intraocular lens wherein the haptics and the optical lens are separate structures. The haptics 14 and 16, as well as other haptics, haptic attachment structures, and haptic-like structure in this and other embodiments are angled anteriorly to provide a posterior vaulting of the optic to eliminate the possibility of chafing or rubbing along the posterior lens surface and to minimize pigment dispersion. The haptics 14 and 16, being those of a one-piece posterior chamber implantable intraocular lens, while being generally continuous, include semi-discontinuous attachment structure in the form of configured recesses 30 and 32 which include slots 34 and 36 which intersect with slots 38 and 40, respectively. The configured recesses 30 and 32 can be any suitable geometrical configuration as long as there are intersecting slots 34 and 38 and intersecting slots 36 and 40 which can engage through enclavement the posterior region of the iris to affix the posterior chamber implantable intraocular lens 10 in the posterior chamber. Such enclavement includes engagement of tissue of the posterior region of the iris. During an enclavement procedure, posterior iris tissue is urged through the slots 38 and 40 and the respective intersecting slots 34 and 36 of the configured recesses 30 and 32. Although the configured recesses 30 and 32 are depicted on the inside aspects 42 and 43 of the haptics 14 and 16, respectively, the configured recesses 30 and 32 could be located elsewhere, such as on the outside aspects 44 and 45 of the haptics 14 and 16, respectively, or at numerous locations between and along the inside aspects 42 and 43 and the outside aspects 44 and 45 of the haptics 14 and 16, as well as any suitable location along or about the haptics 14 and 16, at the junction of the haptics 14 and 16 with the optical lens 12, or along an aspect of the optical lens 12. The posterior chamber implantable intraocular lens 10 can be implanted using conventional implant techniques in the case of a suitable posterior chamber or can be implanted using enclavement techniques if conventional implant techniques cannot be used, such as in the case of a broken or inadequate posterior chamber. The posterior chamber implantable intraocular lens 10 and other posterior chamber implantable intraocular lenses of the invention, shown as alternative embodiments, can be fashioned of suitable polymer material, and can be flexible and foldable, semi-rigid or rigid, or combinations thereof. The posterior chamber implantable intraocular lens 10, and other posterior chamber implantable intraocular lenses shown as other embodiments, are of one-piece design being molded or otherwise fashioned with haptics integral to the optical lens, thereby eliminating the laborious staking of haptics into the optical lens and thus facilitating the manufacturing process as well as providing a suitable and reliable haptic; however, although the practice of staking the haptics to the optical lens could still be utilized in this and other embodiments to provide a posterior chamber implantable intraocular lens of multi-piece design according to the teachings of the invention.

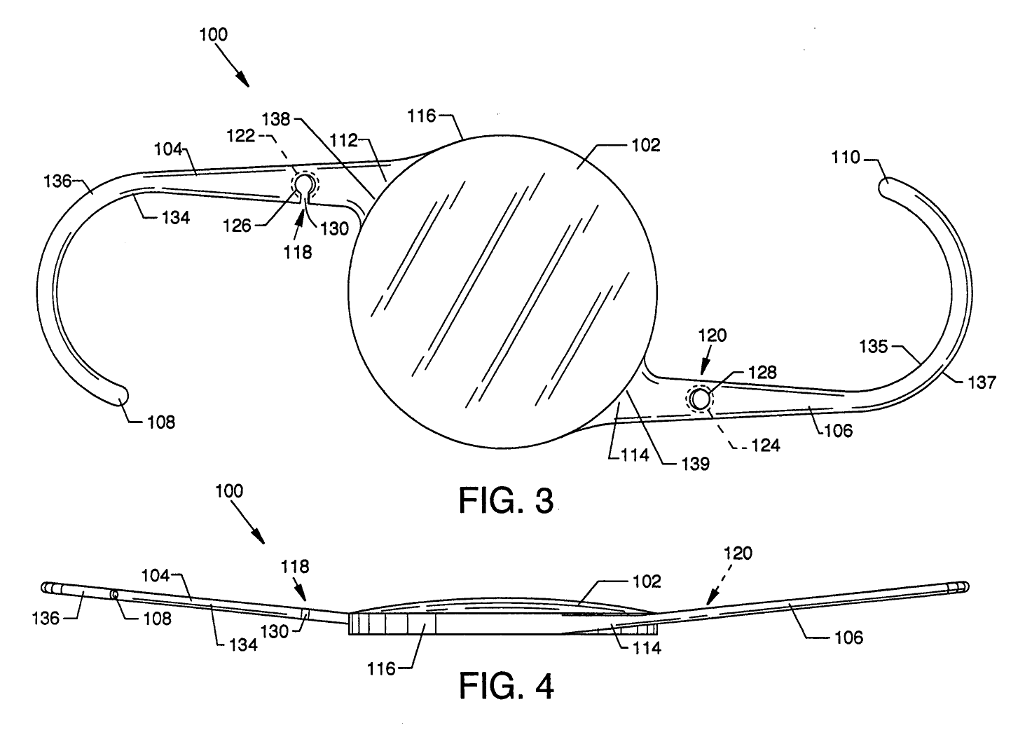

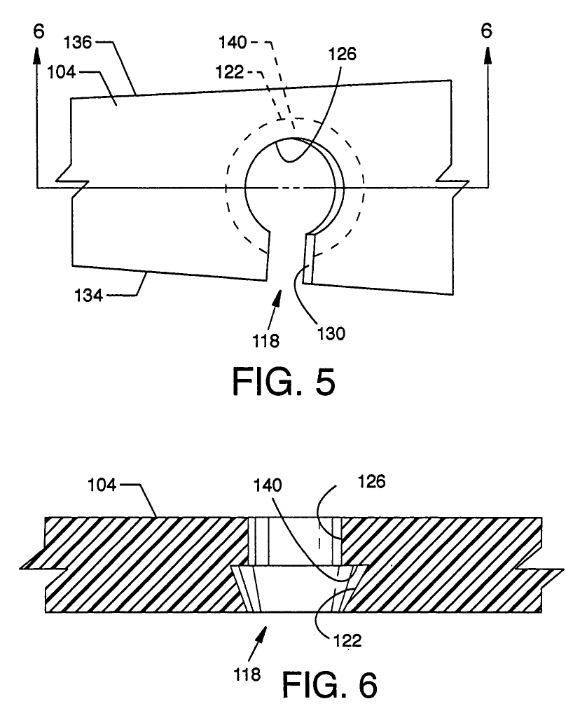

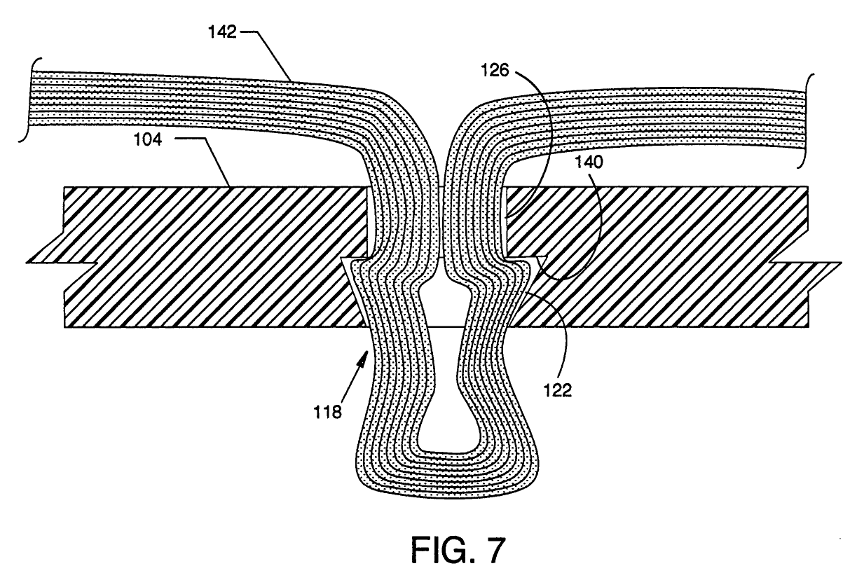

FIG. 3, a first alternative embodiment, is a top view, and FIG. 4 is a side view of a one-piece posterior chamber implantable intraocular lens 100, including an optical lens 102 and diametrically opposed haptics 104 and 106 with outward ends 108 and 110 and integrally constructed inward ends 112 and 114 wherein the inward ends 112 and 114 of the haptics 104 and 106 intersect and are continuous with the periphery 116 of the optical lens 102. The haptics 104 and 106 of the one-piece posterior chamber implantable intraocular lens 100, as illustrated, while being generally continuous, include semi-discontinuous attachment structure in the form of configured recesses 118 and 120 having truncated conical portions 122 and 124 aligned and continuous with cylindrical portions 126 and 128, respectively. In haptic 104, an intersecting slot 130 simultaneously intersects the truncated conical portion 122 and the cylindrical portion 126 in the manner as shown in detail in FIGS. 5 and 6. A slot like slot 130 could be provided at configured recess 120. Alternatively, the slot 130 at configured recess 118 could be eliminated. Thus, the haptics 104 and 106 can be identical or can be different, as shown, depending on whether both, one, or neither of the configured recesses 118 and 120 has an intersecting slot. The configured recesses 118 and 120 can be any suitable geometrical configuration as long as there is suitable structure, such as, but not limited to, the geometrical configuration shown with regard to the configured recesses 118 and 120 which can engage through enclavement the posterior region of the iris to affix the posterior chamber implantable intraocular lens 100 in the posterior chamber. Such enclavement includes engagement of tissue of the posterior region of the iris, as shown in FIG. 7 where during an enclavement procedure, posterior iris tissue is urged through the slot 130 of the configured recess 118 to the interior of the configured recess 118, or can be urged into the interior of the configured recess 120 in the haptic 106. Although the configured recess 118 is depicted opening on the inside aspect 134 of the haptic 104, the configured recess 118 could be located elsewhere, such as where the configured recess 118 opens to the outside aspect 136 of the haptic 104 or at numerous locations between and along the inside aspect 134 and the outside aspect 136 of the haptic 104, as well as any suitable location along or about the haptic 104, at the junction 138 of the haptic 104 with the optical lens 102, or along an aspect of the optical lens 102. In similar manner, the configured recess 120 could also be located elsewhere at numerous locations between and along the inside aspect 135 and the outside aspect 137 of the haptic 106, as well as any suitable location along and about the haptic 106, at the junction 139 of the haptic 106 with the optical lens 102 or along an aspect of the optical lens 102. The posterior chamber implantable intraocular lens 100 can be implanted using conventional implant techniques in the case of a suitable posterior chamber or can be implanted using enclavement techniques if conventional implant techniques cannot be used, such as in the case of a broken or inadequate posterior chamber. The posterior chamber implantable intraocular lens 100 and other posterior chamber implantable intraocular lenses of the invention can be fashioned of a suitable polymer material, and can be flexible and foldable, semi-rigid or rigid, or combinations thereof and can be of one-piece design as shown, but could also be of multiple member construction. Other posterior chamber implantable intraocular lenses shown are of one-piece design being molded or otherwise fashioned with haptics integral to the optical lens, thereby eliminating the laborious staking of haptics into the optical lens and thus facilitating the manufacturing process as well as providing a suitable and reliable haptic; however, the practice of staking the haptics to the optical lens could still be utilized in this and other embodiments to provide a posterior chamber implantable intraocular lens of multi-piece design according to the teachings of the invention.

FIG. 5 is a top view of the configured recess 118 of FIG. 3 showing a portion of the haptic 104. Shown in particular is the alignment of the slot 130 with the combined intersecting and aligned truncated conical portion 122 and cylindrical portion 126.

FIG. 6 is a cross section view of the haptic 104 along line 6-6 of FIG. 5. The alignment of the truncated conical portion 122 with the cylindrical portion 126 is shown in particular. The combination of the truncated conical portion 122 with the cylindrical portion 126 provides structure which is suited for accommodation of iris material, such as shown in FIG. 7. In particular, the area of the truncated conical portion 122 adjacent to an annular shoulder 140 between the truncated conical portion 122 and the cylindrical portion 126 provides an area for fixed accommodation of iris material, as shown in FIG. 7.

FIG. 7 shows the imbrication of iris material 142 in the configured recess 118 of the haptic 104. Iris material 142 is urged first into and then beyond the slot 130 into the aligned truncated conical portion 122 and cylindrical portion 126 to extend beyond the truncated conical portion 122, as shown. The iris material 142 is compacted in the truncated conical portion 122 and cylindrical portion 126 and as such attempts to expand outwardly to fill the allowable space provided by the truncated conical portion 122 and cylindrical portion 126, and in doing so, frictionally engages the walls of the truncated conical portion 122 and cylindrical portion 126, and expands to fixatingly fill the space common to the annular shoulder 140 and the truncated conical portion 122. In addition, the iris material 142 extending through and beyond the truncated conical portion 122 also expands to a profile larger than the outer and smaller extremity of the truncated conical portion 122 to further anchor the iris material 142 to the haptic 104. Capture of iris material 142 by the truncated conical portion 124 and cylindrical portion 128 of the configured recess 120 in the haptic 106 is accomplished in much the same manner without the use of a slot, such as incorporated into use with the configured recess 118.

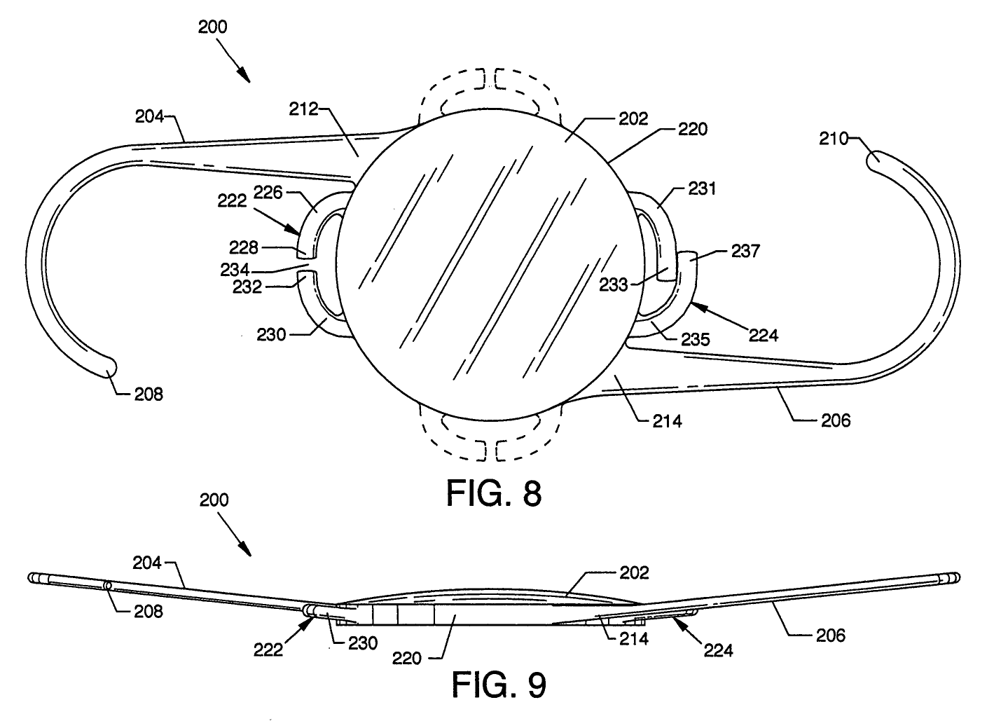

FIGS. 8 and 9, showing a second alternative embodiment, are a top view and a side view of a one-piece posterior chamber implantable intraocular lens 200, including an optical lens 202 and diametrically opposed haptics 204 and 206 with outward ends 208 and 210 and inward ends 212 and 214 wherein the inward ends 212 and 214 of the haptics 204 and 206 are continuous with the periphery 220 of the optical lens 202. Associated with the haptics 204 and 206 is discontinuous attachment structure in the form of engagement fixtures 222 and 224 which extend outwardly from the periphery 220 of the optical lens 202 in the same directions that the haptics 204 and 206 extend and which can engage through enclavement the posterior region of the iris to affix the posterior chamber implantable intraocular lens 200 in the posterior chamber. Such enclavement includes engagement of tissue of the posterior region of the iris. The engagement fixture 222 includes a first extension 226 extending from the optical lens 202 having an end 228 and an opposing second extension 230 extending from the optical lens 202 having an opposed end 232, there being a gap 234 located between the opposed ends 228 and 232 of the opposing first extension 226 and second extension 230. Alternatively, the engagement fixture 224 relatingly depicts a third extension 231 having an end 233 and a fourth extension 235 having an end 237 where the ends 233 and 237 and adjacent regions are horizontally imbricated, i.e., overlap horizontally, as depicted, or alternatively are vertically imbricated, i.e., overlap over and under vertically. Such overlaps can be a contacting overlap where the ends 233 and 237 and near adjacent regions touch each other, or can be a non-contacting overlap where a space exists between the ends 233 and 237 and near adjacent regions, either case being suitable for receipt of iris tissue therebetween. Further, the gap 234 and the imbricated ends 233 and 237 of the third and fourth extensions 231 and 235 are shown in centralized fashion, but could be offset from such central locations toward the junctions formed with the periphery 220 of the optical lens 202. The ends 228, 232, 233 and 237 and adjacent regions including the first extension 226, the second extension 230, the third extension 231, and the fourth extension 235 could also include configured recesses similar to the configured recesses 30 and 32 of FIGS. 1 and 2 dispersed therealong and thereabout, as previously described. Optionally, engagement fixtures similar to engagement fixtures 222 and 224 could be disposed transverse to the haptics 204 and 206; that is, located at the twelve o'clock and six o'clock positions about the periphery 220 of the optical lens 202, as depicted in dashed lines in FIG. 8. During an enclavement procedure, posterior iris tissue is urged through the gap 234 to engage the first and second extensions 226 and 230. The optical lens 202 and the haptics 204 and 206 and the engagement fixtures 222 and 224 can be formed of a suitable polymer. The posterior chamber implantable intraocular lens 200 can be implanted using conventional implant techniques in the case of a suitable posterior chamber or can be implanted using enclavement techniques if conventional implant techniques cannot be used, such as in the case of a broken or inadequate posterior chamber. The posterior chamber implantable intraocular lens 200 embodiment, like other embodiments of the invention, is foldable, and the foldable attributes of the posterior chamber implantable intraocular lens 200 and other embodiments allow manual placement whereby the unfolding of the posterior chamber implantable intraocular lens 200 provides for locating the posterior chamber implantable intraocular lens 200 in the posterior chamber without requiring enclavement techniques. Such foldable design allows placement which is more facile, practical or perhaps even automatic, in that, during the unfolding deployment of the invention, iris material can be automatically engaged.

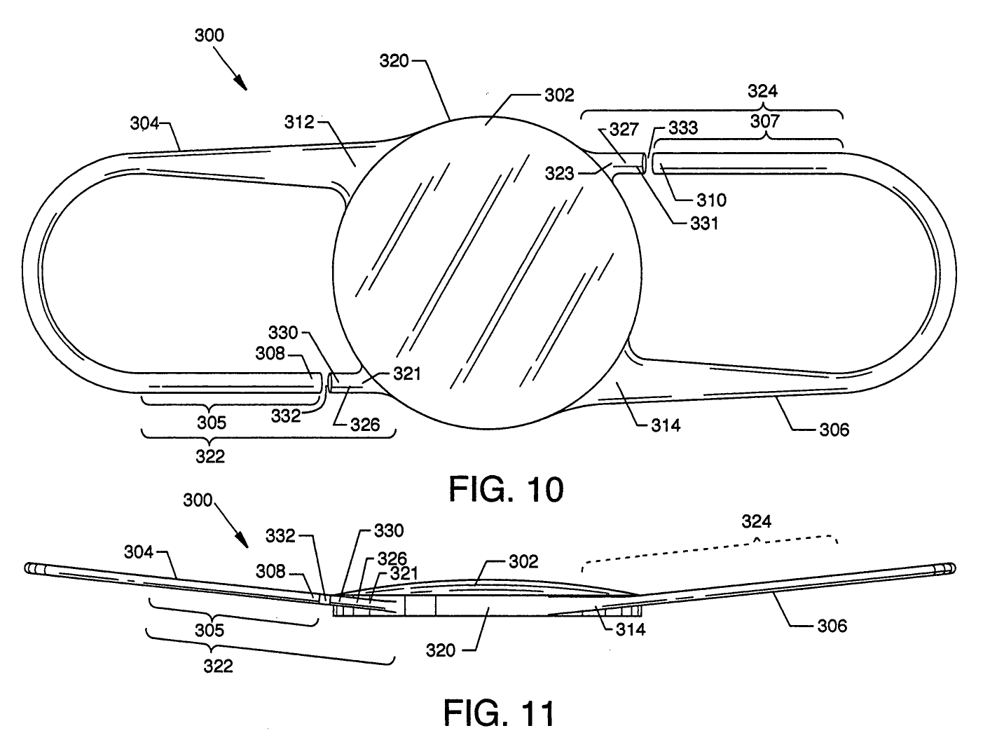

FIGS. 10 and 11, showing a third alternative embodiment, are a top view and a side view of a one-piece posterior chamber implantable intraocular lens 300, including an optical lens 302 and diametrically opposed haptics 304 and 306 having extended portions 305 and 307 terminating in outward ends 308 and 310 and having inward ends 312 and 314 continuous with the periphery 320 of the optical lens 302. Associated with the haptics 304 and 306 is discontinuous attachment structure in the form of opposed and elongated engagement fixtures 322 and 324 of similar design and function which extend outwardly from the periphery 320 of the optical lens 302, a portion of each of which can engage through enclavement the posterior region of the iris to affix the posterior chamber implantable intraocular lens 300 in the posterior chamber. Such enclavement includes engagement of tissue of the posterior region of the iris. The engagement fixture 322 includes the extended portion 305 of the haptic 304, including the outward end 308, a projection 326 having an outward end 330, an inward end 321 continuous with the periphery 320 of the optical lens 302, and a discontinuity in the form of a gap 332 located between the outward end 308 of the extended portion 305 of the haptic 304 and the opposing outward end 330 of the projection 326. The engagement fixture 324 is of similar design and function with respect to the engagement fixture 322 and includes the extended portion 307 of the haptic 306, including the outward end 310, a projection 327 having an outward end 331, an inward end 323 continuous with the periphery 320 of the optical lens 302, and a discontinuity in the form of a gap 333 located between the outward end 310 of the extended portion 307 of the haptic 306 and the opposing outward end 331 of the projection 327. The gaps 332 and 333 are shown in a location near the optical lens 302, but alternatively could be located more greatly distanced from the optical lens 302. In the alternative, the outward ends 330 and 331 of the projections 328 and 327 could be lengthened to extend outwardly in horizontal or vertical imbrication beyond the outward ends 308 and 310 of the extended portions 305 and 307 of the haptics 304 and 306, respectively, to provide discontinuous overlapping structure. During an enclavement procedure, posterior iris tissue is urged through the gaps 332 and 333 to engage the extended portions 305 and 307 of the haptics 304 and 306 and the projections 326 and 327, or to engage discontinuous imbricated overlapping structure where posterior iris tissue is urged through, around and about the discontinuous overlapping imbricated structure, if provided. The optical lens 302 and the haptics 304 and 306 including the extended portions 305 and 307 and the projections 326 and 327 can be formed of a suitable polymer. The posterior chamber implantable intraocular lens 300 can be implanted using conventional implant techniques in the case of a suitable posterior chamber or can be implanted using enclavement techniques if conventional implant techniques cannot be used, such as in the case of a broken or inadequate posterior chamber.

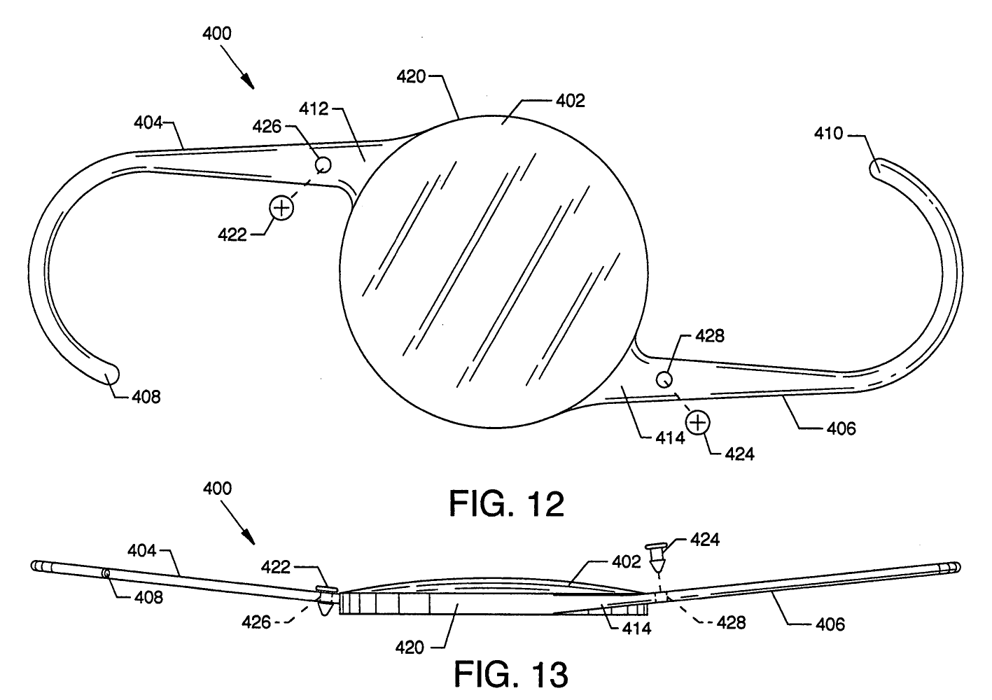

FIGS. 12 and 13, showing a fourth alternative embodiment, are a top view and a side view of a posterior chamber implantable intraocular lens 400, including an optical lens 402 and diametrically opposed haptics 404 and 406 with outward ends 408 and 410 and inward ends 412 and 414 wherein the inward ends 412 and 414 of the haptics 404 and 406 are continuous with the periphery 420 of the optical lens 402. The haptics 404 and 406 have semi-discontinuous attachment structure resulting from holes 426 and 428 being provided therethrough for the receipt of fasteners 422 and 424. When enclavement of the tissue of the iris is not feasible, attachment of the posterior chamber implantable intraocular lens 400 can be accomplished by the use of the fasteners 422 and 424, which can be miniature barbed clips, miniature nut and bolt assemblies, or capturing head fasteners. Fasteners 422 and 424 can pass through the iris to secure through the holes 426 and 428 in the haptics 404 and 406. The optical lens 402 and the haptics 404 and 406 and the fasteners 422 and 424 can be formed of a suitable polymer. The posterior chamber implantable intraocular lens 400 can be implanted using conventional implant techniques in the case of a suitable posterior chamber or can be implanted using the above fastener technique if conventional implant techniques cannot be used, such as in the case of a broken or inadequate posterior chamber.

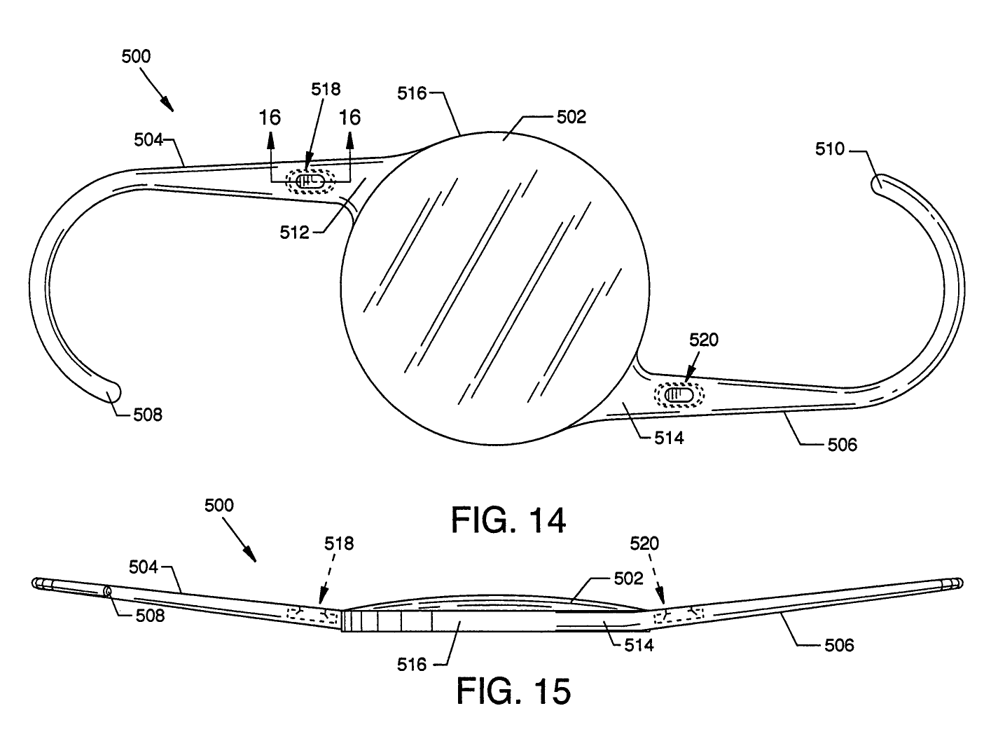

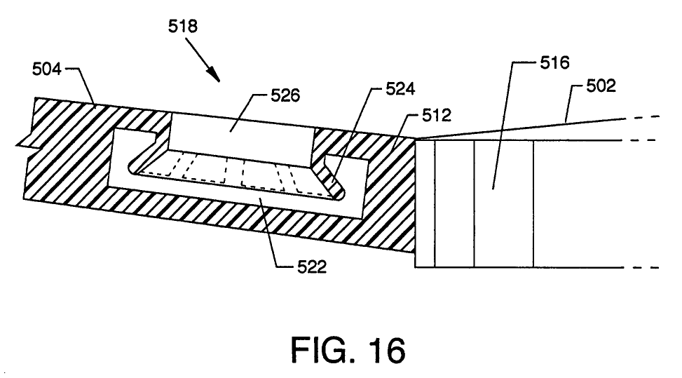

FIGS. 14 and 15, showing a fifth alternative embodiment, are a top view and a side view of a posterior chamber implantable intraocular lens 500, including an optical lens 502 and diametrically opposed haptics 504 and 506 with outward ends 508 and 510 and inward ends 512 and 514 wherein the inward ends 512 and 514 of the haptics 504 and 506 are continuous with the periphery 516 of the optical lens 502. The haptics 504 and 506 have semi-discontinuous attachment structure resulting from configured recesses 518 and 520 being provided therein for the reception of iris tissue. The configured recesses 518 and 520 are identical and as such only the structure of the configured recess 518 is described with reference to FIG. 16, which is a cross section view taken along line 16-16 of FIG. 14. The configured recess 518 includes a cavity 522 of suitable or desired shape internal to the haptic 504 and an internal flange 524 which extends into the cavity 522 and which can be angled with respect to the cavity 522. An orifice 526 extends from the outer surface of the haptic 504 and through the internal flange 524 for access into the cavity 522 and for passage and imbrication of iris material. The internal flange 524 can be of continuous circumference or can be segmented or interrupted as shown by dashed lines to provide structure to enclave, snag, hook or otherwise capture iris tissue. Alternatively, tab-like structure could be provided in lieu of the internal flange 524. Attachment of the posterior chamber implantable intraocular lens 500 can be accomplished by the use of the configured recesses 518 and 520. Optionally, the haptics 504 and 506 can be thickened to more readily accommodate iris material. The optical lens 502 and the haptics 504 and 506 can be formed of a suitable polymer. The posterior chamber implantable intraocular lens 500 can be implanted using conventional implant techniques in the case of a suitable posterior chamber or can be implanted using the above enclavement technique involving configured recesses 518 and 520 if conventional implant techniques cannot be used, such as in the case of a broken or inadequate posterior chamber.

Various modifications can be made to the present invention without departing from the apparent scope thereof.

### PARTS LIST

10 posterior chamber implantable intraocular lens
12 optical lens
14 haptic
16 haptic
18 outward end
20 outward end
22 inward end
24 inward end
29 periphery
30 configured recess
32 configured recess
34 slot
36 slot
38 slot
40 slot
42 inside aspect
43 inside aspect
44 outside aspect
45 outside aspect
100 posterior chamber implantable intraocular lens
102 optical lens
104 haptic
106 haptic
108 outward end
110 outward end
112 inward end
114 inward end
116 periphery
118 configured recess
120 configured recess
122 truncated conical portion
124 truncated conical portion
126 cylindrical portion
128 cylindrical portion
130 slot
134 inside aspect
135 inside aspect
136 outside aspect
137 outside aspect
138 junction
139 junction
140 annular shoulder
142 iris material
200 posterior chamber implantable intraocular lens
202 optical lens
204 haptic
206 haptic
208 outward end
210 outward end
212 inward end
214 inward end
220 periphery
222 engagement fixture
224 engagement fixture
226 first extension
228 end
230 second extension
231 third extension
232 end
233 end
234 gap
235 forth extension
237 end
300 posterior chamber implantable intraocular lens
302 optical lens
304 haptic
305 extended portion
306 haptic
307 extended portion
308 outward end
310 outward end
312 inward end
314 inward end
320 periphery
321 inward end
322 engagement fixture
323 inward end
324 engagement fixture
326 projection
327 projection
330 outward end
331 outward end
332 gap
333 gap
400 posterior chamber implantable intraocular lens
402 optical lens
404 haptic
406 haptic
408 outward end
410 outward end
412 inward end
414 inward end
420 periphery
422 fastener
424 fastener
426 hole
428 hole
500 posterior chamber implantable intraocular lens
502 optical lens
504 haptic
506 haptic
508 outward end
510 outward end
512 inward end
514 inward end
516 periphery
518 configured recess
520 configured recess
522 cavity
524 internal flange
526 orifice

It is claimed:

1. A posterior chamber implantable intraocular lens comprising:
a. an optical lens, the optical lens having a periphery;
b. diametrically opposed haptics extending from the periphery of the optical lens; and,
c. attachment structure associated with the diametrically opposed haptics.

2. The posterior chamber implantable intraocular lens of claim 1, wherein the attachment structure associated with the diametrically opposed haptics is semi-discontinuous.

3. The posterior chamber implantable intraocular lens of claim 2, wherein each haptic has an outward end.

4. The posterior chamber implantable intraocular lens of claim 3, wherein the semi-discontinuous attachment structure comprises a configured recess of each haptic situated on the haptic between the outward end of the haptic and the periphery of the optical lens.

5. The posterior chamber implantable intraocular lens of claim 4, wherein each configured recess includes intersecting slots.

6. The posterior chamber implantable intraocular lens of claim 4, wherein each haptic has an inside aspect and an outside aspect, and wherein the configured recess of each haptic is located on the inside aspect.

7. The posterior chamber implantable intraocular lens of claim 4, wherein each haptic has an inside aspect and an outside aspect, and wherein the configured recess of each haptic is located on the outside aspect.

8. The posterior chamber implantable intraocular lens of claim 4, wherein each haptic has an inside aspect and an outside aspect, and wherein the configured recess of each haptic is located between the inside aspect and the outside aspect.

9. The posterior chamber implantable intraocular lens of claim 4, wherein each haptic forms a junction with the periphery of the optical lens, and wherein the configured recess of each haptic is located at the junction.

10. The posterior chamber implantable intraocular lens of claim 4, wherein each configured recess includes a truncated conical portion intersecting a cylindrical portion.

11. The posterior chamber implantable intraocular lens of claim 10, wherein a slot extends from an aspect of at least one haptic to the configured recess of that haptic and intersects both the truncated conical portion and the cylindrical portion of the configured recess.

12. The posterior chamber implantable intraocular lens of claim 4, wherein each configured recess comprises a cavity internal to the haptic, an internal flange extending into the cavity, and an orifice extending from the outer surface of the haptic through the internal flange and into the cavity.

13. The posterior chamber implantable intraocular lens of claim 12, wherein the internal flange is angled with respect to the orifice.

14. The posterior chamber implantable intraocular lens of claim 12, wherein the internal flange has a continuous circumference.

15. The posterior chamber implantable intraocular lens of claim 12, wherein the internal flange is segmented.

16. The posterior chamber implantable intraocular lens of claim 2, wherein the semi-discontinuous attachment structure includes a mounting hole through each haptic for accepting a fastener, and wherein a fastener is included for each mounting hole.

17. The posterior chamber implantable intraocular lens of claim 16, wherein each fastener is selected from the group consisting of a barbed clip, a nut/bolt assembly, and a capturing head fastener.

18. The posterior chamber implantable intraocular lens of claim 16, wherein each haptic includes a widened portion through which the mounting hole for accepting a fastener is formed.

19. The posterior chamber implantable intraocular lens of claim 1, wherein the attachment structure associated with the diametrically opposed haptics is discontinuous.

20. The posterior chamber implantable intraocular lens of claim 19, wherein the discontinuous attachment structure comprises an engagement fixture.

21. The posterior chamber implantable intraocular lens of claim 20, wherein the engagement fixture is located on the periphery of the optical lens and comprises first and second extensions extending outwardly from the periphery, the first and second extensions having opposed ends spaced apart and defining a gap therebetween.

22. The posterior chamber implantable intraocular lens of claim 20, wherein the engagement fixture is located on the periphery of the optical lens and comprises first and second extensions extending outwardly from the periphery, the first and second extensions having opposed ends which overlap horizontally.

23. The posterior chamber implantable intraocular lens of claim 20, wherein the engagement fixture is located on the periphery of the optical lens and comprises first and second extensions extending outwardly from the periphery, the first and second extensions having opposed ends which overlap vertically.

24. The posterior chamber implantable intraocular lens of claim 20, wherein the engagement fixture comprises an extended portion of a haptic terminating in an outward end, a projection extending outwardly from the periphery of the optical lens, and a gap between the outward end and the projection.

25. The posterior chamber implantable intraocular lens of claim 19, wherein the discontinuous attachment structure comprises two engagement fixtures located on the periphery of the optical lens.

26. The posterior chamber implantable intraocular lens of claim 25, wherein the two engagement fixtures are aligned with the diametrically opposed haptics.

27. The posterior chamber implantable intraocular lens of claim 25, wherein the two engagement fixtures are disposed transverse to the diametrically opposed haptics.

28. The posterior chamber implantable intraocular lens of claim 19, wherein the discontinuous attachment structure comprises two engagement fixtures, each engagement fixture comprising an extended portion of a haptic terminating in an outward end, a projection extending outwardly from the periphery of the optical lens, and a gap between the outward end and the projection.

29. The posterior chamber implantable intraocular lens of claim 1, wherein the optical lens has mounting holes in the periphery and each haptic has inward ends secured in a mounting hole.

30. The posterior chamber implantable intraocular lens of claim 1, wherein the haptics are formed of polymer material.

31. The posterior chamber implantable intraocular lens of claim 1, wherein the haptics and the optical lens are formed of polymer material.

32. The posterior chamber implantable intraocular lens of claim 1, wherein the optical lens and the haptics are a one-piece integral structure.

33. The posterior chamber implantable intraocular lens of claim 1, wherein the diametrically opposed haptics are angled anteriorly.

34. A posterior chamber implantable intraocular lens comprising:
a. an optical lens, the optical lens having a periphery;
b. diametrically opposed haptics extending from the periphery of the optical lens; and,
c. discontinuous attachment structure associated with the haptics in the form of opposed engagement fixtures extending from the periphery of the optical lens, the opposed engagement fixtures allowing for attachment of posterior iris tissue through enclavement.

35. The posterior chamber implantable intraocular lens of claim 34, wherein each opposed engagement fixture includes a first extension and a second extension, each of the extensions having an end opposed to the end of the other extension.

36. The posterior chamber implantable intraocular lens of claim 35, wherein the opposed ends of the extensions define a gap.

37. The posterior chamber implantable intraocular lens of claim 35, wherein the opposed ends of the extensions are horizontally imbricated.

38. The posterior chamber implantable intraocular lens of claim 35, wherein the opposed ends of the extensions are vertically imbricated.

39. The posterior chamber implantable intraocular lens of claim 35, wherein the extensions include configured recess each including intersecting slots.

40. The posterior chamber implantable intraocular lens of claim 34, wherein the opposed engagement fixtures are aligned with the diametrically opposed haptics.

41. The posterior chamber implantable intraocular lens of claim 34, wherein the opposed engagement fixtures are disposed transverse to the diametrically opposed haptics.

42. The posterior chamber implantable intraocular lens of claim 34, wherein all parts thereof are formed of polymer material.

43. The posterior chamber implantable intraocular lens of claim 34, wherein all parts thereof are formed as a one-piece construction.

44. A posterior chamber implantable intraocular lens comprising:
a. an optical lens, the optical lens having a periphery;
b. diametrically opposed haptics extending from the periphery of the optical lens, each of the haptics having an extended portion terminating in an outward end; and,
c. projections extending from the periphery of the optical lens, each of the projections having an end, each end being located near an outward end of an extended portion of a haptic and defining a gap therewith, and wherein the diametrically opposed haptics including the extended portions with outward ends together with the projections define a pair of opposed and elongated engagement fixtures.

45. A method of installing a posterior chamber implantable intraocular lens in a posterior chamber posterior to iris tissue, the method comprising the steps of:
a. providing a posterior chamber implantable intraocular lens, the posterior chamber implantable intraocular lens including an optical lens having a periphery, diametrically opposed haptics extending from the periphery, and attachment structure associated with each haptic;
b. determining the suitability and adequacy of the posterior chamber for conventional implantation; and,
c. installing the provided posterior chamber implantable intraocular lens in the posterior chamber by conventional implantation if the step of determining reveals the posterior chamber to be suitable and adequate for conventional implantation, or installing the provided posterior chamber implantable intraocular lens in the posterior chamber by attaching it to posterior iris tissue using the attachment structure if the step of determining reveals the posterior chamber to be unsuitable and inadequate for conventional implantation.

46. The method of claim 45, wherein the step of determining the suitability and adequacy of the posterior chamber for conventional implantation includes a determination of whether the posterior chamber is broken.

47. The method of claim 45, wherein the step of installing the posterior chamber implantable intraocular lens is by conventional implantation subsequent to determining the posterior chamber to be suitable and adequate for conventional implantation.

48. The method of claim 45, wherein the step of installing the posterior chamber implantable intraocular lens is by using the attachment structure subsequent to determining the posterior chamber to be unsuitable and inadequate for conventional implantation.

49. The method of claim 48, wherein the step of installing includes an enclavement procedure.

50. The method of claim 48, wherein the step of installing includes a fastening procedure.
